(19) United States
(12) Patent Application Publication
(10) Pub. No.: US 2016/0270767 A1
(43) Pub. Date: Sep. 22, 2016

(54) Spessel Urine Collection Device

(71) Applicant: Naomi Jessica Travers, Poway, CA (US)

(72) Inventor: Naomi Jessica Travers, Poway, CA (US)

(21) Appl. No.: 15/068,543

(22) Filed: Mar. 12, 2016

Related U.S. Application Data
(60) Provisional application No. 62/132,230, filed on Mar. 12, 2015.

(51) Int. Cl. A61B 10/00

(52) U.S. Cl. CPC A61B 10/007

(57) ABSTRACT

The present invention provides a generally hollow vessel which articulates with the female genitalia and, when left in situ, functions to opportunistically collect urine when the patient inevitably voids her bladder. The device is further designed to sequester the first portion of said urine, which tends to be unsuitable for diagnostic testing, and then, divert the remainder of relatively uncontaminated urine, preferably into a collection receptacle where it can be easily accessed for testing.

### FIELD OF THE DISCLOSURE

The present invention is in the field of medical devices. More particularly, the present invention is in the field of urine collection devices and, more particularly still, in the field of urine collection for the purpose of obtaining a sample for diagnostic purposes. More particularly, the present invention is in the field of urine sample collection from physically and/or cognitively impaired individuals.

### BACKGROUND OF THE INVENTION

The collection of a urine sample to test for infection and/or renal function is one of the most common diagnostic tests required in the health care arena. It would be difficult to estimate how many thousands of such tests are performed on a daily basis. Coupled with assessment of clinical symptoms, a clean, ideally sterile sample of urine is the cornerstone of proper diagnosis, treatment decision-making and therapeutic evaluation of antibiotic effectiveness for the management of urinary tract infection (UTI).

When patients present with common symptoms of a UTI a urine sample is typically obtained in order to perform a simple test with a diagnostic “dipstick” which detects certain markers that indicate a UTI. Based on those preliminary results, the health care practitioner will often treat empirically for a UTI with appropriate antibiotics as established by accepted guidelines for the most likely causative organisms. A urine sample is then sent for further laboratory studies—either a urinalysis and/or a culture to verify the causative organism and the antibiotic therapy to which it is most sensitive. Antibiotic therapy may be continued or changed based on this information. A secondary urine sample may be obtained at later date after therapy has finished to confirm that the infection is in fact gone.

Currently there are several methods for collecting a sample, which can be as little as 10 to 20 milliliters for proper testing. For unimpaired individuals, it is standard to have them sit on a toilet, urinate a small portion and then place the urine collection container in the path of the urine stream to get a “clean catch” sample.

However, this seemingly simple task for some is either very time consuming or completely impossible for others due to a variety of physical and cognitive impairments. For the ambulant elderly, the act of maneuvering into the bathroom and stooping onto the toilet can be slow and painful. In the hospital setting, patients may be immobile, unconscious or impaired in some other fashion such that they cannot provide a urine sample in the traditional manner. Elderly patients may be incontinent and not able to control their urine, making the prospect of providing a urine sample unpredictable and time-consuming.

The nursing home patient poses an especially difficult population with regard to urine sampling. They are often bedridden and may have dementia so that they cannot or will not follow the commands to provide a sample. It is well documented that the incidence of urinary tract infections increases with age. Incontinence, impaired immunity and anatomical changes, particularly in females, make the aged population increasingly susceptible to UTIs. Unlike younger and healthy individuals, the aged patient may not present with the typical symptoms of painful urination, lower abdominal pain or, in the case of severe upper tract infection, fever. Even if these symptoms are experienced, the demented patient is often unable to communicate them. Often change in mental status and/or behavior in the form of an acute delirium may be the only sign of infection. While UTI is a possible cause of delirium, it is only one of many considerations. The delirious patient often requires a urine sample to rule out UTI as a cause of their altered mental status, but it is this status that renders them unable to provide the diagnostic sample to help improve their condition.

Another common method of obtaining a urine sample is to insert a catheter directly into the bladder. However, this process is invasive and disturbing to most patients, especially the very old and the very young. It is time-consuming for staff and may be impossible to conduct in the aggressive and confused patient. Lastly, a sample may be obtained by inserting a needle into the abdomen at the location of the bladder and aspirating urine directly. This procedure also has obvious drawbacks and is a last resort.

Nursing staff may, for lack of an alternative, put a urinalysis dipstick onto a urine-soaked diaper from a compromised patient, in order to get preliminary information on urinary/renal status. However, this source is likely to be contaminated and does not yield a fluid sample as required by the laboratory to do advance testing. Similarly, nursing staff may attempt to have patients void on a bedpan. However, this can be a cumbersome and time consuming process for a patient who has trouble lifting their hips onto the bedpan or who, for cognitive reasons, cannot understand that they are meant to urinate into the pan in a timely fashion.

In view of the difficulties in getting a urine sample from patients, providers may opt to empirically treat a suspected urinary tract infection with antibiotics. Ideally this is followed by a culture and sensitivity test as previously mentioned. This test, which requires a clean urine sample, is essential for establishing the causative organism. Without this follow up, the provider is forced to gamble on antibiotics therapy.

In order to compensate for the inability to verify the causative organism, and to increase the chances of treatment for the supposed UTI, providers may opt to treat with broad-spectrum antibiotics. These are agents that are effective against multiple organisms. This practice constitutes an inappropriate and misguided use of antibiotics and is a major contributor to the growing problem of antibiotic resistance.

Antibiotic resistance is such a threat to health care globally that the World Health Organization (WHO) dedicated its 2011 World Health Day to highlighting the problem and establishing suggested guidelines to minimize it.

There is a clear need for another alternative to the current urine collection methods, which are inadequate for the population that has a particularly high demand for this diagnostic procedure. While there is extensive prior art in the field of urine collection, a significant portion is meant to manage incontinence by sequestering large volumes of urine with the intention of discarding it. As such, the cleanliness, sterility and quality of collected urine are inconsequential and these devices are designed accordingly.

There is also a notable body of prior art in the field of devices which aim to collect a midstream urine sample from the female population. Implicit in the design and description of these embodiments is the assumption that users are cognitively able to identify when they need to urinate and to communicate this need effectively to caregivers, and that users are physically able to maneuver the pelvis over a toilet or at least a bedpan.

The large number of devices relating to urine collection reflects the importance of the genitourinary system to human health, and the complexity of related pathologies which necessitate medical interaction with this system. Depending on the ultimate purpose of a particular device, significant differences in male and female anatomy often necessitate completely different embodiments for each sex. Recognizing the difficulty of obtaining midstream urine sample, even in able-bodied patients, there are several devices designed to divide the urine stream and divert the relatively contaminant-free urine which presumably represents a true sample from the bladder.

U.S. Pat. No. 4,457,314, entitled “Non-invasive urine collection device for females” represents early prior art in the field that attempts to provide a collection device that can be used with impaired patients. The present invention targets the same population subset.

U.S. Pat. No. 3,680,543, entitled “Urine collection device” is one comparative device which intends to divert the first portion of urine from the second portion of expelled urine. The device description suggests that the diversion mechanism be manually activated and thus, would require a level of dexterity and cognitive awareness not required by the present invention.

Similarly, U.S. Pat. No. 8,328,733, a “Urine collection device” aims to divide the first and second portions of urine flow via an inner mechanism, with a funnel-shaped receptacle as the receiving aperture. This funnel component does not suitably minimize contact of urine with the external genitalia, and as such is likely to produce a lesser-quality sample as compared to the present invention. U.S. Pat. No. 4,494,581, entitled “Isolation of forestream and midstream portions of collected urine samples” has a similar problem with the quality of the sample obtained.

Furthermore, the shape, size and configuration of the embodiments in the above-mentioned patents appear to be impractical for use with a bedbound patient, who is mostly likely supine or partially reclined. All appear to be too bulky to fit inside a diaper or incontinence pad and would seem to require that either the patient or nurse hold the device in place, presumably over the toilet.

U.S. Pat. No. 3,750,647, entitled “Means for collecting specimens of urine” relies on the presumed differentiation of flow trajectories of initial versus midstream urine flow to separate a suitable sample. Like U.S. Pat. No. 3,680,543, the invention requires manual manipulation during the procedure to engage the diversion mechanism. As such, it requires involvement from the patient and/or caregiver, which the present invention eliminates.

U.S. Pat. No. 8,202,263, a “Vaginal Barrier and female Urethral Catheterization Device” incorporates a vaginal insertion protrusion that is similar to embodiments of the present invention; however, the invention differs significantly in other respects.

U.S. Pat. No. 4,911,698 demonstrates a “Clean-Catch Intra-labia Urinary Collection Device,” which, in recognition of the need to separate the labia minora to minimize contamination, describes an ovoid opening that encircles the urinary meatus. This feature is similar to the receiving orifice of the present invention. However, U.S. Pat. No. 4,911,698 lacks any diversion mechanism and dependable attachment method. As such, it would require significant intra-procedural manipulation by either the patient and/or nurse in order to complete the midstream urine collection process.

U.S. Pat. No. 6,923,755, entitled “Stimulator” teaches the concept of a labial attachment appendage. The invention uses a clip assembly to attach a clitoral stimulation device. The clip assembly employs a spring that is under constant tension when it is attached to the user.

In contrast to the previously referenced embodiments, the present invention aims to collect a midstream urine sample from the bedbound and/or cognitively impaired patient. It is uniquely designed to overcome these impairments with regard to the process of collecting a midstream urine sample, such that it can be used in the bedbound patient without intra-procedural manipulation by the patient or nurse/caregiver. As such, it teaches a novel product not found in the current body of prior art.

An express purpose of the present invention is to provide a device that can adapt to the cognitive and physical limitations of a frail, elderly population whereupon patients are often demented, physically incapacitated and/or bedbound such that their participation in the urine collection process is extremely limited. This subset of the female population is scarcely addressed by the existing body of related prior art. The uniquely designed shape and mechanisms of the present invention reflect its novel intention and distinguish it from other embodiments in the field.

### SUMMARY OF THE INVENTION

The present invention consists of a generally hollow vessel which articulates or couples with the female exterior genitalia and, when left in situ, functions to opportunistically collect urine when the patient inevitably voids her bladder. Preferably, the device, through an anchor, only attaches or couples to the genetalia of the female person from whom the urine sample is to be collected. This makes the procedure for placing the collection vessel simple and the female person does not have to be moved or overly bothered. The device is further designed to sequester the first portion of said urine, which tends to be unsuitable for diagnostic testing, and then, divert the remainder of relatively uncontaminated urine, preferably into a collection receptacle where it can be easily accessed for testing.

The device is generally defined by the following major components: first, a receiving opening or orifice is provided that articulates with the female genitals and allows urine to flow into a hollow, interior collection chamber. This chamber encloses a simple mechanism or reservoir that sequesters approximately the first 20 milliliters of urine. The remaining uncontaminated urine can be diverted into an adjacent collection receptacle. Preferably the collection receptacle is easily visualized and disconnected to access the desired urine sample. Another major component is an anchor. In one preferred embodiment, the anchor is a moveable, winged appendage which can be closed around the outside of the labia majora such that it secures the collection chamber within the labia majora.

The cross-sectional shape of the collection vessel is that of a relatively thin ellipse which allows the device to sit comfortably between the legs, parallel to the sagittal anatomic plane. In the preferred embodiment, the main body measures approximately 11 centimeters in height, approximately 1.5 centimeters in width and four centimeters in depth.

On the anteior aspect of the collection vessel, which faces the patient, is a preferably elliptically shaped opening that extends approximately four centimeters from the top of the device to just above midlevel. This opening generally mimics the size and shape of the female vaginal introitus, allowing it to articulate closely with the genital tissue, to sit within the labia minora and to encircle the urinary meatus. In the preferred embodiment, this opening measures approximately four centimeters in length by one centimeter in width. The placement of the rim circumference within the labia minora lends an important feature to the device; that of minimizing contact of urine with genital tissue. The urinary meatus is unencumbered and expelled urine may flow freely into the hollow inner chamber below. Around the rim of the device opening is a preferably soft, pliable cushion, which further enhances comfort and conformity to individual anatomy.

In certain embodiments, an anchor is provided just below the bottom edge of the receiving opening as a bulbous projection approximately 1 cm in diameter and 1 cm in depth. This anchor can be used in combination with the moveable, winged appendage anchor or separately. This projection serves as an anatomical anchor and marker; when placed within the vaginal orifice, it ensures the proper placement of the receiving opening around the urinary meatus, as female genitourinary anatomy is universal in its placement of the urinary meatus directly above the vaginal opening.

In certain embodiments, just distal to the lower rim of the opening, preferably attached to the opposite, posterior portion of interior wall of the collection vessel and attached laterally to the interior wall of the chamber, is a hooded structure which slants obliquely downward. Below this structure is a preferably circular opening henceforth known as the “inferior opening.” A pair of openings, henceforth known as the “superior openings,” are located opposite each other on the lateral portions of the interior wall of the chamber, above the inferior opening. A distance of approximately one centimeter separates the inferior opening and the pair of superior openings, such that back flowing urine, to be described later, will preferentially flow first into the inferior opening and into the collection bag rather than into the superior openings. The superior openings serve as an overflow outlet, should the volume of collected urine exceed the capacity of the chamber.

The shape and placement of the hooded structure, coupled with gravitational forces, compels the urine to flow downward while bypassing the shielded inferior opening underneath. The inside of the chamber is preferably lined with or made of any number of hydrophobic materials or substances, thus creating a repelling force that enhances the tendency of the first flow of urine to pool at the bottom of the chamber.

Preferably, the bottom of the device houses an entrapment mechanism or reservoir that is a mass of fine, liquid-absorbing substance, preferably a powder, and preferably polyacrylate. The powder is preferably enclosed by a flexible, porous envelope such that it allows urine to pass through while particles of powder, either in dry form or engorged with fluid, stay contained within. Preferred materials for encasing the powder would be any paper derivative, fabric or fine mesh.

The fineness of the powder presents a collectively large surface area such that urine is instantaneously absorbed on contact. The mass of powder is sufficient to absorb approximately 20 milliliters of urine before the resulting gelatinous mass reaches capacity. This first 20 milliliters of urine approximates the volume that sits in the urethra, which is contaminated with epithelial cells and must be eliminated from urine in the bladder to avoid a false-positive diagnostic study.

Against the gelatinous barrier, the remainder of forthcoming urine fills the chamber. As the fluid level rises to the level of the inferior opening, the urine must exit the chamber via a flexible tube which is connected to a preferably pliable plastic container. In the preferred embodiment, this container sits outside of the patient's diaper or incontinence pad, allowing for easy visualization and access by the nurse or caregiver.

The final component is a winged appendage anchor that serves to anchor the inner collection chamber in place. A secure attachment method is an important design element of the device, as this eliminates the need for intra-procedural manipulation by either the patient or caregiver.

The present invention is intended to optimally serve the purpose of collecting urine in its cleanest form directly after being expelled from the bladder; to minimize contact with surrounding genital skin while avoiding invasion into the urinary tract; to sequester said urine and, finally, to allow easy and sterile transfer of urine into a standard testing vessel.

The novel design of the present invention allows the device to function irrespective of the patient's physical and/or cognitive capacity to participate in the process. Indeed, the patient may be a totally passive participant while the device compensates for the impairments of frail, bedbound female patients who may be unable to comprehend the task and/or lack the physical agility to comply with the traditional urine collection method.

### DESCRIPTION OF DRAWINGS

FIG. 1 is a perspective view of an embodiment of the present invention. Interior components of the invention, including the hood and the liquid absorbing material and the envelope that contains it are shown through the wall of the collection vessel without the use of dotted lines.

FIG. 2 shows an embodiment of the present invention in situ.

FIG. 3 shows an isometric view of an alternate embodiment of the present invention with the winged attachment appendage used with the collection vessel.

FIG. 4 shows a detailed, partial sectional view of a ratchet mechanism used with the winged attachment appendage of the present invention.

FIG. 5 shows a superior of the vessel and winged attachment appendage of one embodiment of the present invention.

FIG. 6 shows the embodiment of the present invention with winged attachment appendage in situ.

FIG. 7 shows an alternative attachment structure. The hood structure is shown in the interior of the collection vessel, although not in dotted lines.

### DETAILED DESCRIPTION OF THE INVENTION

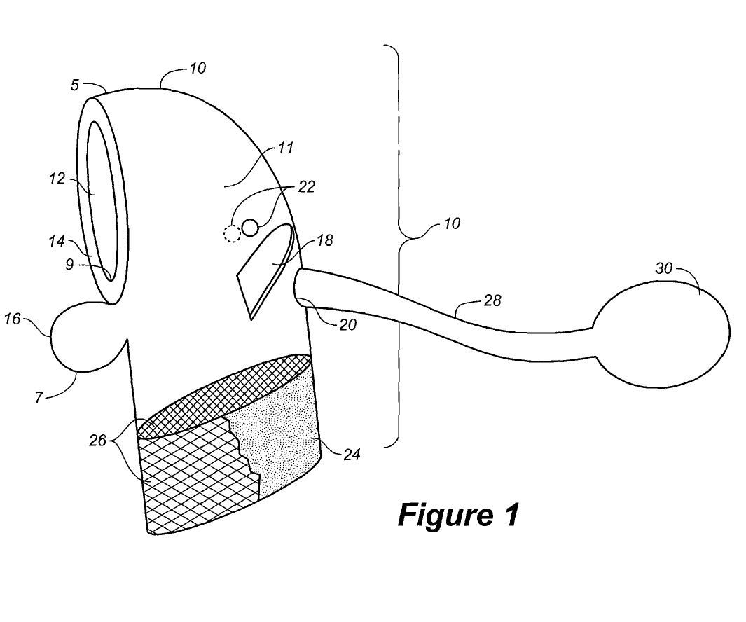

FIG. 1 shows the collection vessel 10. On the superior-anterior aspect of the collection vessel 10 is the receiving opening orifice 12, which preferably sits between the labia minora 1 and encircles the urinary meatus 3. The cross-sectional shape of the collection vessel 10 is that of a relatively thin ellipse which allows the device to sit comfortably between the legs, parallel to the sagittal anatomic plane. In the preferred embodiment, the collection vessel 10 measures approximately 11 centimeters in height, approximately 1.5 centimeters in width and four centimeters in depth.

On the anterior aspect of the collection vessel 10, which faces the patient, the receiving orifice 12 is a preferably elliptically shaped opening that extends approximately four centimeters from the top 5 of the collection vessel 10 to just above midlevel. This receiving opening 12 generally mimics the size and shape of the female vaginal introitus, allowing it to articulate closely with the genital tissue, to sit within the labia minora 1 and to encircle the urinary meatus 3. In the preferred embodiment, the receiving opening 12 measures approximately four centimeters in length by one centimeter in width. The placement of the rim circumference of receiving opening 12 within the labia minora 1 lends an important feature to the device; that of minimizing contact of urine with genital tissue. The urinary meatus 3 is unencumbered and expelled urine may flow freely into the hollow inner chamber 11 of the collection vessel 10.

Around the rim of the receiving opening 12 is a preferably soft, pliable cushion 14, which further enhances comfort and conformity to individual anatomy. The cushion 14 is made of soft, preferably malleable material and surrounds the receiving orifice 12 and functions both as a comfortable cushion and a mediator between the collection vessel 10 and individual tissue anatomy.

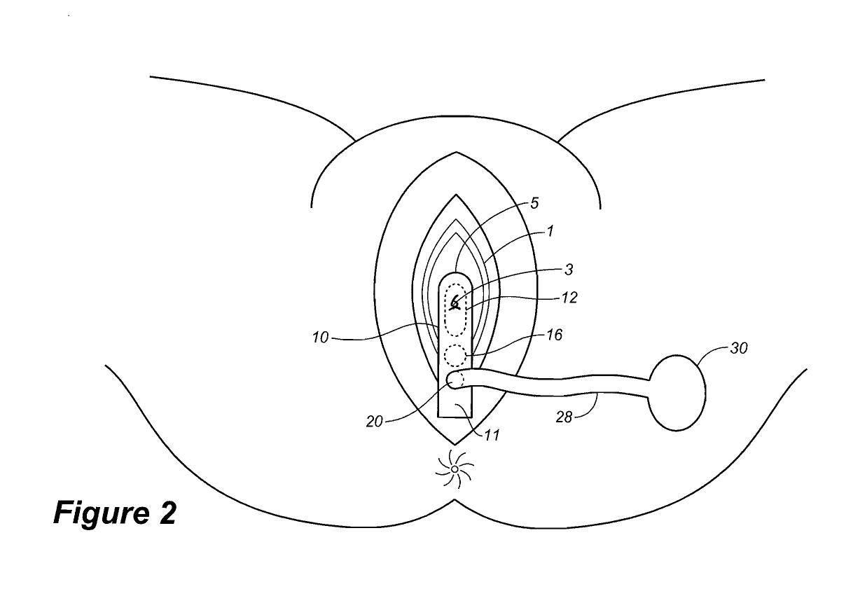

In certain embodiments, an anchor 7 is provided just below the bottom edge 9 of the receiving opening as a soft, bulbous projection or protrusion 16. As shown in FIG. 1, the protrusion 16 is approximately 1 cm in diameter and 1 cm in depth. A shown in FIG. 2, the soft, bulbous protrusion 16 sits in the vaginal orifice or opening and serves as an anchor 7. This anchor 7 can be used in combination with the moveable, winged appendage anchor 13 or separately. This projection 16 serves as an anatomical anchor 7 and marker; when placed within the vaginal orifice, it ensures the proper placement of the receiving opening 12 around the urinary meatus 3, as female genitourinary anatomy is universal in its placement of the urinary meatus 3 directly above the vaginal opening 15.

As urine is expelled, the fluid may flow unencumbered via the receiving opening 12 and into the inner chamber 11 of the collection vessel 10. A slanted hood or projection 18, preferably located on the interior of the dorsal or posterior wall shields an inferior opening 20 which is also, preferably located on the posterior wall of the collection vessel 10 at generally the same elevation and slightly below where the uppermost portion of the hood 18 contacts the wall of the collection vessel 10. The hood 18 is preferably located at an elevation just below the lower rim 9 of the receiving opening 12, preferably on the opposite, posterior wall. The sides of the hood 18 are preferably attached to the lateral walls of collection vessel 10 and it slants obliquely downward into the interior of the chamber 11, thus preventing the urine stream from entering the inferior opening 20 directly.

Preferably, the collection vessel 10 is made with two, superior twin openings 22 on either lateral side of the collection vessel 10 that act as overflow drainage portals. The orientation and exact location of the hood 18, the inferior opening 20, and the one or more superior openings 22 of the collection vessel 10 may vary and still serve the same functions. More important to the function of the inferior and superior openings 20 and 22 is their differential height whereby the inferior opening 20 is significantly lower than the one or more superior openings 22 such that back flowing urine will naturally exit the inferior opening 20 and thus fill the collection receptacle 30. Gravitational forces compel the fluid to flow over the hood 18, to bypass the opening 20, and ultimately collect at the bottom of the collection vessel 10. At the bottom of the vessel 10 is an entrapment reservoir or mechanism which is preferably a mass of liquid-absorbing material 24, preferably a powder, encased in an envelope 26 that is porous. The liquid-absorbing material 24 is a preferably fine powder, and preferably polyacrylate. The powder 24 is preferably enclosed by a flexible, porous envelope 26 such that it allows urine to pass through while particles of powder, either in dry form or engorged with fluid, stay contained within. Preferred materials for encasing the powder would be any paper derivative, fabric or fine mesh.

Upon contact, this liquid absorbing material or powder 24 instantaneously absorbs approximately 20 milliliters of urine before reaching capacity and redirecting subsequent urine upward into the interior chamber 11 of the collection vessel 10 until the fluid level reaches the inferior opening 20. As the patient continues to expel urine, the volume overflows via the inferior opening 20 and into a tube 28, which is connected to a collection receptacle 30, which is preferably a bag.

This urine is relatively free of contaminants, which have been sequestered within the first portion of urine absorbed by the liquid-absorbing powder 24. Any urine that exceeds the capacity of the collection bag 30 will exit via the superior openings 22 and into a diaper or incontinence pad (not shown).

FIG. 2 demonstrates the invention in situ. The receiving aperture 12, represented by dotted lines, sits between the labia minora 1 and encircles the urinary meatus 3. The anchoring protrusion 16, also represented by dotted lines, sits inside the vaginal orifice.

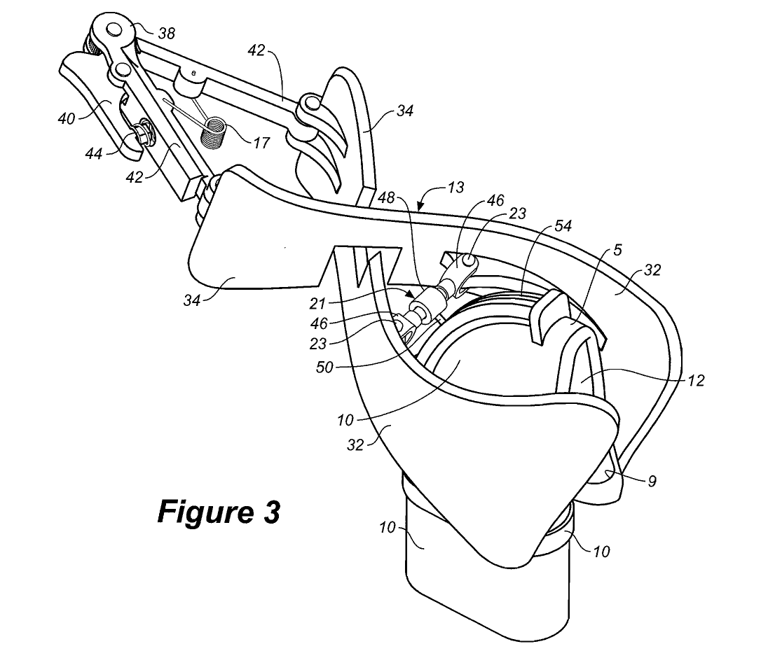

FIG. 3 is an isometric view of one embodiment of the present invention with the winged appendage anchor 13 or attachment mechanism for coupling to the female person's genetalia. The winged appendage anchor 13 is attached to the dorsal “spine” of the collection vessel 10. The wings intersect and rotate with respect to each other in the same fashion as a common pair of scissors. This configuration creates two opposing sets of levers, inner levers 32 and outer levers 34 which, preferably are either biased apart and so have an open resting position or are not biased to either open or close. However, while it is not preferred, the levers could be biased by action of a spring to a closed position. The ends of the inner levers 32 are smooth and align anatomically with the base of the labia majora 15 where the labia majora 15 meets the groin. When the outer levers 34 are compressed, the inner levers 32 simultaneously close. A ratchet assembly attached to the outer levers 34 allows the inner levers 32 to close incrementally according to individual anatomy and comfort. When the receiving orifice 12 is place around the urinary meatus 3, the outer levers 34 are compressed and simultaneously compel the inner levers 32 toward each other, similar to the cutting action of scissors. The levers closest to the receiving orifice 12 are the inner levers 32.

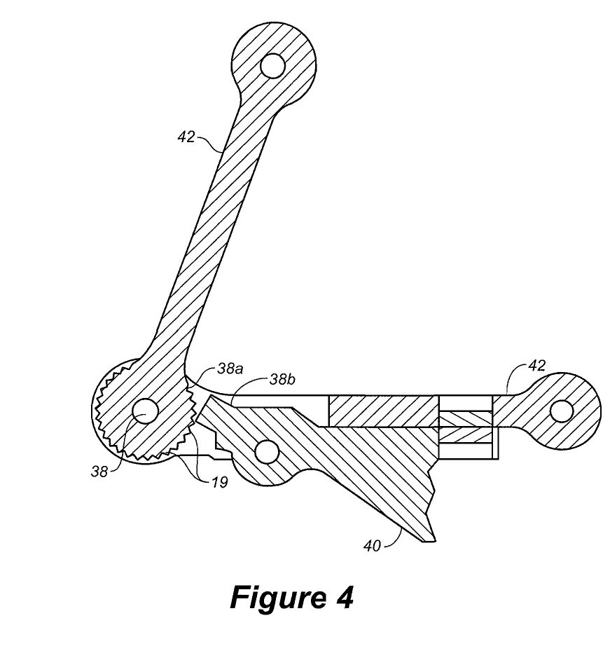

As shown in FIG. 4, in one embodiment of the invention a simple ratchet mechanism is provided between outer levers 34, which allows the inner levers 32 to be closed incrementally. In the preferred embodiment, the ratchet mechanism consists of a saw-toothed wheel 38a and pawl 38b attached at the ends of ratchet levers 42. The ratchet mechanism is similar to that commonly used in many simple tools. Saw-toothed wheel 38a is located within joint 38 between ratchet levers 42 and is part of one of the ratchet levers 42. Pawl 38b at the end of pawl lever 40 is rotatingly attached to the other of the ratchet levers 42. When the two outer levers 34 are compressed, the angle between ratchet levers 42 is reduced simultaneously moving the saw-toothed wheel 38a with respect to the pawl 38b. As shown in FIG. 4, toothed wheel 38a is at the end of one of the ratchet levers 42 and, as shown, is not a complete circle or wheel. Rachet levers 42 are joined at wheel 38a and joint 38. Ratchet levers 42 are pivotly attached to outer levers 34. Inward movement or closing of the outer levers 34, produces inward movement or closing of the ratchet levers 42 which produces a torque force that rotates the wheel 38a with respect to pawl 38b which is pivotly attached to the other ratchet lever 42. Counter tension is provided by a tension spring 17 between the ratchet levers 42 that pushes the ratchet levers 42 apart and the teeth 19 of the wheel 38a against the pawl 38b. Movement of the outer levers 34 by the operator overcomes the biasing force exerted by the tension spring 17. Tension spring 17 could also act directly on outer levers 34. FIG. 3 also shows a ratchet spring 44 which exerts a counterforce against the pawl 38b and prevents it from slipping backward, thus stabilizing the wheel 38a incrementally in its position between each notch of the teeth 19. Pawl lever arm 40 may be released, overcoming the action of spring 44, to free the pawl 38b and thus open the ratchet levers 42 and the outer and inner levers 34 and 32 for removal of the device.

As the user incrementally compresses the outer levers 34, the inner levers 32 simultaneously close around the outside of the labia majora 15 in a similarly incremental manner, thus stabilizing the collection vessel 10. Both the shape of the inner levers 32 and the variable pressure against the labia majora 15 contribute to firm but comfortable attachment of the entire device. The aforementioned ratchet mechanism may be replaced by any number of similar mechanisms, of various configurations and at different locations between the lever components 32 and 34 that could serve the purpose of allowing incremental compression of the inner levers around the labia majora 15.

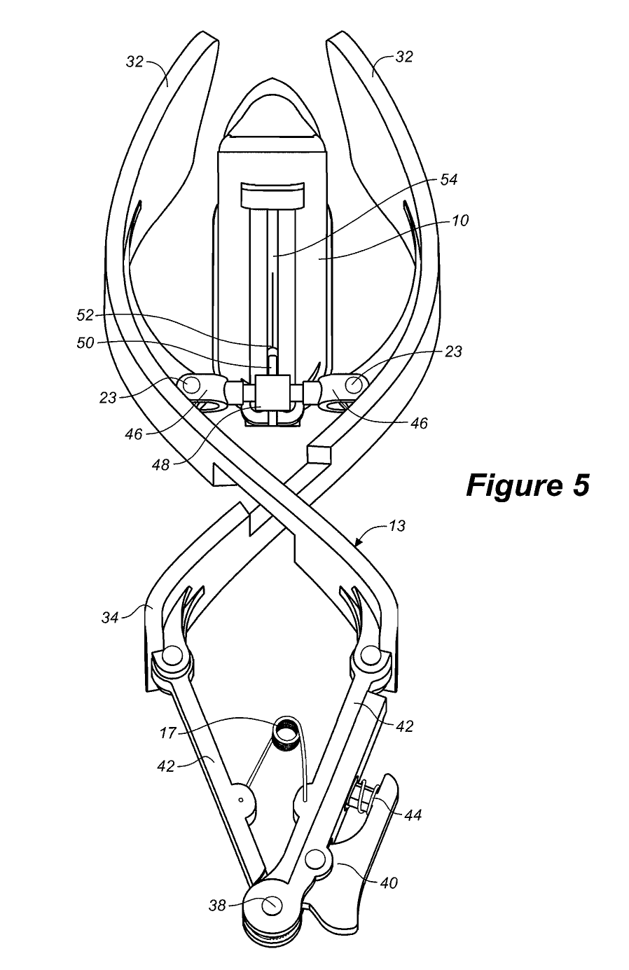

FIG. 5 shows the articulated bridge 21 with hinged arms 46, on which the inner levers 32 move in concert with manipulation of the outer levers 34. The bridge 21 articulates with each inner lever 32 via a hinge 23; the hinge 23 allows the inner levers 32 to close when the ratchet is engaged. From the center of the bridge 21 a stem 50 extends downward toward the dorsal spine of the collection vessel 10 and terminates in a locking protrusion 52, preferably a sphere. The locking protrusion 52 sits within a track 54 that runs along the dorsal spine of the collection vessel 10 and allows the winged appendage anchor 13 to move along the track 54; the ball joint created by the spherical locking protrusion 52 allows for three dimensional manipulation of the winged appendage anchor 13. Hinged arms 46 are rotatingly connected to a central fixture 48. A stem 50 extends from the central fixture 48 and terminates in a locking protrusion 52, which slides within the confines of a track 54 formed on the containment vessel 10. These features allow the user to move the attachment and optimize placement of the collection vessel 10 based on individual anatomy and the individuals position.

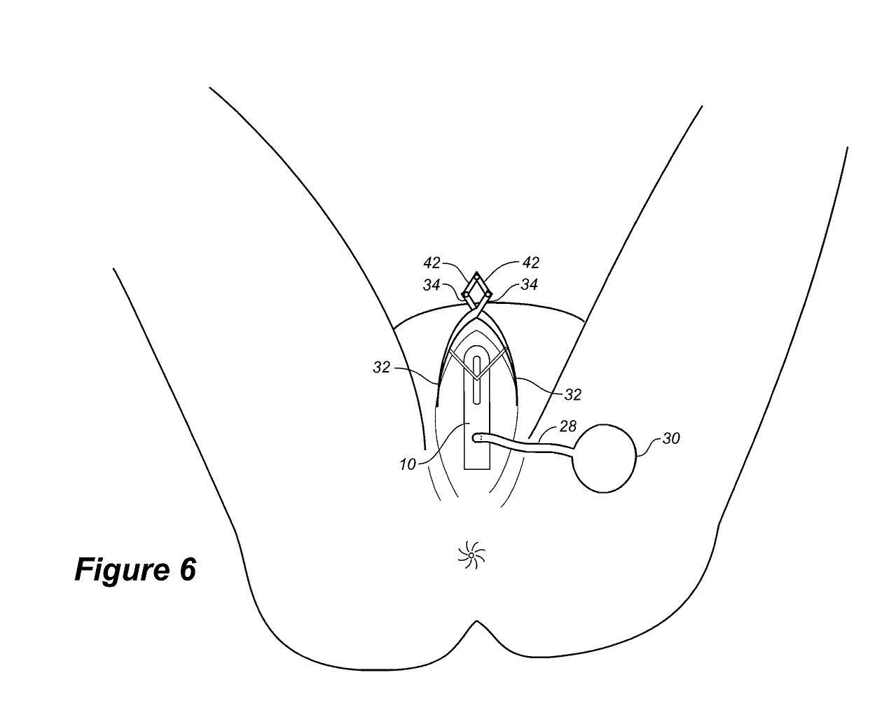

FIG. 6 shows the inner lever 32 closed around the outside of the labia majora 15 and the collection vessel 10 disposed within the labia minora 1.

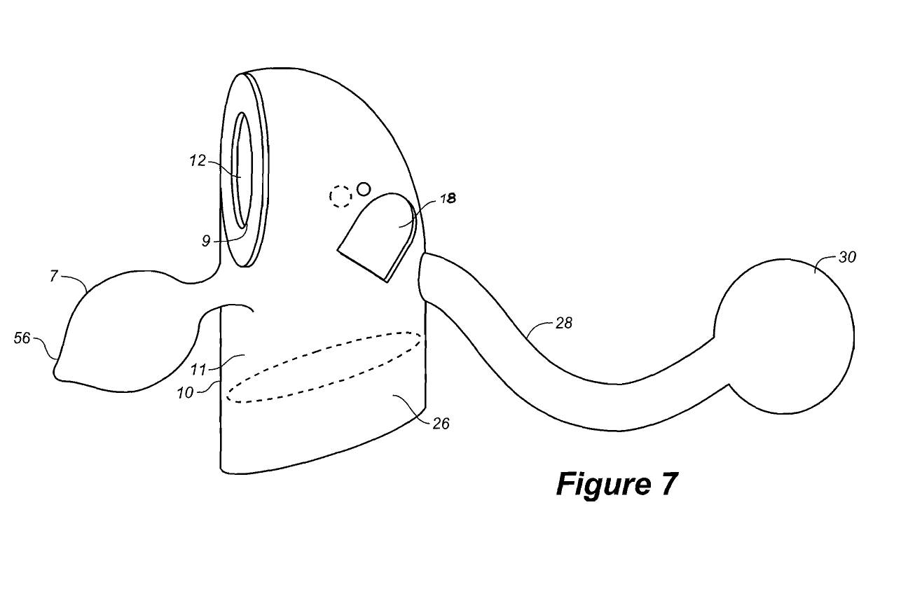

As shown in FIG. 7, in lieu of the preferred winged appendage anchor, or in concert therewith, the collection vessel 10 may be stabilized with an intra-vaginal extension 56 of the previously described bulbous protrusion. This structure is a preferably soft protrusion consisting of a stalk approximately 2 cm in length. This stalk terminates in a preferably bulbous structure which is larger in diameter than the stalk, such that it becomes anchored when inserted into the vagina and enveloped by its soft tissue, coupling the collection vessel 10 to the female person's genetalia.

The central vessel 10 is preferably made by any variety of soft, medical grade rubber, such as silicon. Alternative materials, particularly those that mimic the composition of silicon, but which are biodegradable and/or have other advantageous qualities, are not excluded. The levers are preferably made from a durable plastic or metal.

1. A device for collecting a urine sample from a female person, the female person having genitalia including labia majora and a vaginal opening, the device comprising:
a. a collection vessel having an inner chamber, a receiving orifice that allows entry of urine into the collection vessel and the inner chamber of the collection vessel, and where a portion of the inner chamber is provided with an entrapment reservoir, and
b. the collection vessel has an anchor that couples the collection vessel to the genetalia of the female person

2. The device of claim 1, wherein:
the anchor is provided on the collection vessel and engages the vaginal opening of the female person.

3. The device of claim 1, wherein:
the collection vessel is provided with an inferior opening through which urine can leave the collection vessel, and the inferior opening is provided above the entrapment reservoir, and below the receiving orifice.

4. The device of claim 3, further comprising:
a collection receptacle is provided that is in communication with the collection vessel through the inferior opening.

5. The device of claim 3, wherein:
the collection vessel is provided with a hood that shields the inferior opening and prevents urine entering the collection vessel through the receiving orifice from exiting through the inferior opening without first contacting the inner chamber.

6. The device of claim 1, wherein:
the collection vessel attaches only to the genitalia of the female person providing the urine sample.

7. The device of claim 1, wherein:
the inner chamber of the collection vessel is hydrophobic.

8. The device of claim 1, wherein:
a. the receiving orifice is formed on the anterior portion of the collection vessel, and
b. the anterior portion of the collection vessel on which the receiving orifice is disposed is sufficiently narrow to fit between the labia majora.

9. The device of claim 8, wherein:
a. the collection vessel is generally the same width as the anterior portion on which the receiving orifice is formed;
b. the collection vessel is taller than the collection vessel is wide.

10. The device of claim 1, wherein:
one or more levers are attached to the collection vessel that engage the labia majora to attach the collection vessel to the female person.

11. The device of claim 10, wherein:
the one or more levers are provided with a ratchet mechanism so that the one or more levers may be moved and held in a given position with respect to the collection vessel incrementally.

12. The device of claim 1, wherein:
the entrapment reservoir is a liquid-absorbing material.

13. The device of claim 12, wherein:
the liquid-absorbing material is provided at the bottom of the inner chamber.

14. The device of claim 12, wherein:
the liquid-absorbing material is contained within a porous envelope.

15. The device of claim 12, wherein:
an anchor is provided on the collection vessel that engages the vaginal opening of the female person.

16. The device of claim 12, wherein:
the collection vessel is provided with an inferior opening through which urine can leave the collection vessel, and the inferior opening is provided above the entrapment reservoir, and below the receiving orifice.

17. The device of claim 16, wherein:
the collection vessel is provided with a hood that shields the inferior opening and prevents urine entering the collection vessel through the receiving orifice from exiting through the inferior opening without first contacting the inner chamber.

18. The device of claim 12, wherein:
the inner chamber of the collection vessel is hydrophobic.

19. The device of claim 12, wherein:
a. the receiving orifice is formed on the anterior portion of the collection vessel, and
b. the anterior portion of the collection vessel on which the receiving orifice is disposed is sufficiently narrow to fit between the labia majora.

20. The device of claim 12, wherein:
one or more levers are attached to the collection vessel that engage the labia majora to attach the collection vessel to the female person.
